(19) United States
(12) Plant Patent Application Publication
(10) Pub. No.: US 2001/0042258 P1
(43) Pub. Date: Nov. 15, 2001

(54) New kiwi plant entitled 'Hortgem Tahi'

(76) Inventors: Mark A. McNeilage, Palmerston North (NZ); Ron A. Beatson, Palmerston North (NZ); Elspeth A. MacRae, Palmerston North (NZ)

Correspondence Address: CHRISTIE, PARKER & HALE, LLP, 350 WEST COLORADO BOULEVARD, SUITE 500, PASADENA, CA, 91105, US

(21) Appl. No.: 09/780,809

(22) Filed: Feb. 9, 2001

(30) Foreign Application Priority Data
Feb. 11, 2000 (NZ) KIWI017

(51) Int. Cl. A01H 5/00

(52) U.S. Cl. PLT/156

(57) ABSTRACT

A new and distinct kiwi plant of the species Actinidia arguta (Sieb & Zucc.) Planch. ex Miq. var. arguta Li is described. The variety results from a controlled pollenation using a female A. arguta selection AA02₁₃ 01 of unknown parentage and a male A. arguta selection AA13₁₃ 01 of unknown parentage. Both named parents (AA02-01 and AA13-01) are unpatented. Its green hairless, edible skin, small fruit size and sweet aromatic taste distinguish the new variety.

The benefits under 35 U.S.C. §119 are claimed with respect to New Zealand PVR Application No. KIW017, filed on Feb. 11, 2000 in New Zealand.

### BACKGROUND OF THE INVENTION

Kiwi plants in cultivation are mainly varieties of A. deliciosa, a particularly ‘Hayward’ although some A. chimensis and A. arguta varieties are grown. A. deliciosa and A. chinensis are closely related, whereas A. arguta is classified in a separate section of the genus. A. deliciosa and A. chinensis varieties have large fruit (˜100 g) with hair on the skin. The main varieties in New Zealand are ‘Hayward’ (A. deliciosa) and ‘Hort16A’ (A. chinensis). Fruit are usually cut and eaten with a spoon. A arguta has small fruit (˜10 g) with no hair on the skin. The skin is edible so these fruit can be eaten whole, like a grape.

All Actinidia species are dioecious, so female varieties have to be inter-planted with male pollenizers to ensure fruit production.

A. arguta vines are deciduous and tend to grow vigorously in spring and summer when rapidly-growing shoots can intertwine and tangle if not managed. Vines do best in a mild temperate climate without late spring or early autumn frosts. They produce consistent heavy crops when grown in well-drained fertile soils and given regular irrigation in dry spells.

A. arguta flowers in spring (late October-early December) in New Zealand. Harvest of A. arguta fruit may occur between early February and late March in New Zealand depending on the selection and location of plantings. Compared to A. deliciosa and A. chinensis, A. arguta fruit require more careful handling during harvest and post-harvest procedures.

### SUMMARY OF THE INVENTION

The present invention is a new and distinctive kiwi plant having a small, generally spheroid shaped fruit with green hairless edible skin. This new variety is designated ‘HORTGEM TAHI’ and is derived from a controlled pollination of AA02-01, a female A. arguta selection of unknown parentage, with AA13-01, a male A. arguta selection of unknown parentage.

The female parent was introduced as a plant from England to New Zealand in 1955. The male parent was introduced as scionwood from Scotland to New Zealand in 1982. The provenance of both is unknown. Both parents are unpatented.

This new variety was created during the course of a plant breeding program, which was initiated during 1987 at HortResearch in Auckland, New Zealand. The cross was made in November 1987. Seeds were sown in autumn (March) 1988 and 129 seedlings from this cross were planted out in the field at Kumeu Research Orchard in spring (October) 1988. The seedlings first fruited in approximately February to March 1991. 20 promising female seedlings were clonally propagated into a two-site replicated trial in 1995 and ‘HORTGEM TAHI’ (breeding code K2D4) was selected after storage and sensory evaluation in 1998.

The new variety can be asexually reproduced as cuttings or by grafting or budding on to seedling or cutting-grown rootstocks of A. arguta. Trial plantings as cuttings established in 1995 at Te Puke and Nelson Research Centres and on seedling rootstocks established in 1998 at these sites have shown that the unique combination of characters come true to form and are established and transmitted through succeeding asexual propagation.

‘HORTGEM TAHI’ is a different species to both the unpatented ‘Hayward’ variety and the patented ‘Hort16A’ variety, so their pollenizers cannot be used. Three new and unpatented A. arguta male pollenizers known currently by their breeding codes as B4G4, E4H4 and K1J6, have been selected as males for use in new plantings of ‘HORTGEM TAHI’.

### BRIEF DESCRIPTION OF ILLUSTRATIONS

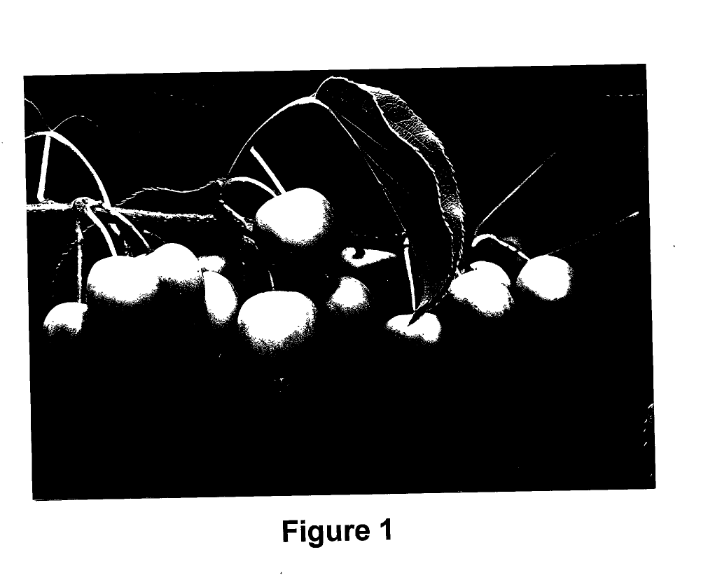

FIG. 1 shows typical fruit of the new variety in the orchard.

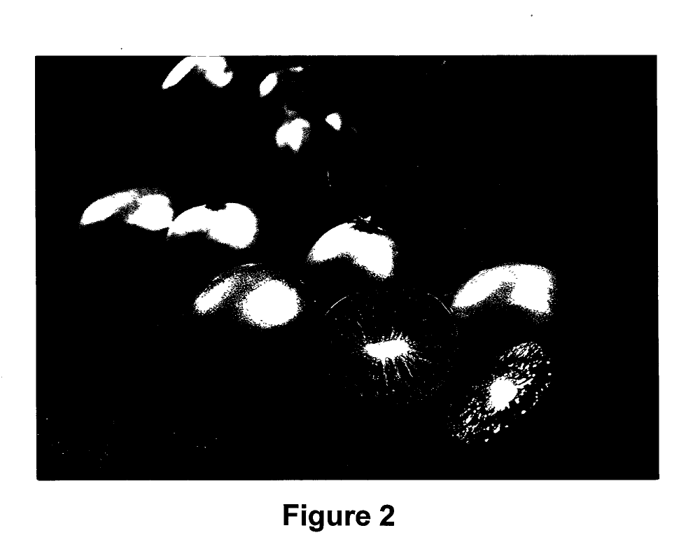

FIG. 2 shows typical fruit of the new variety in the studio.

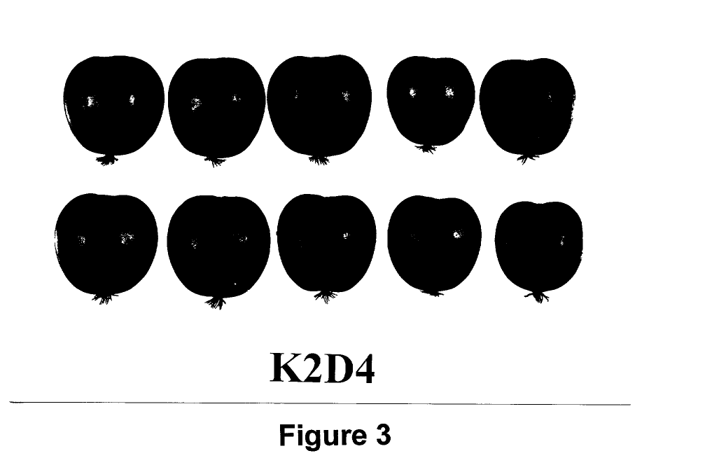

FIG. 3 shows fruit of the new variety in profile.

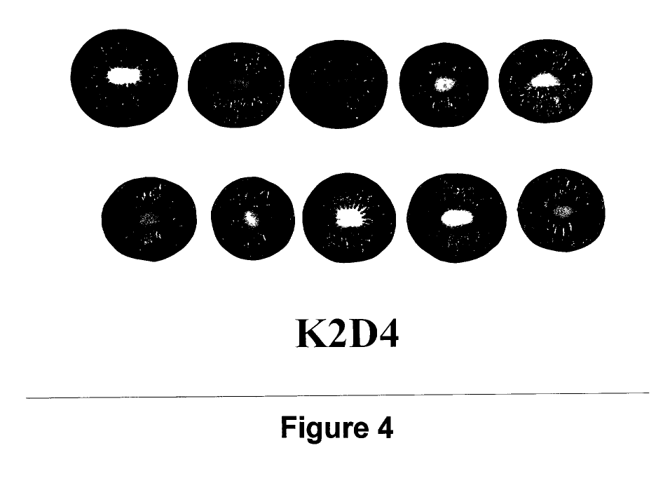

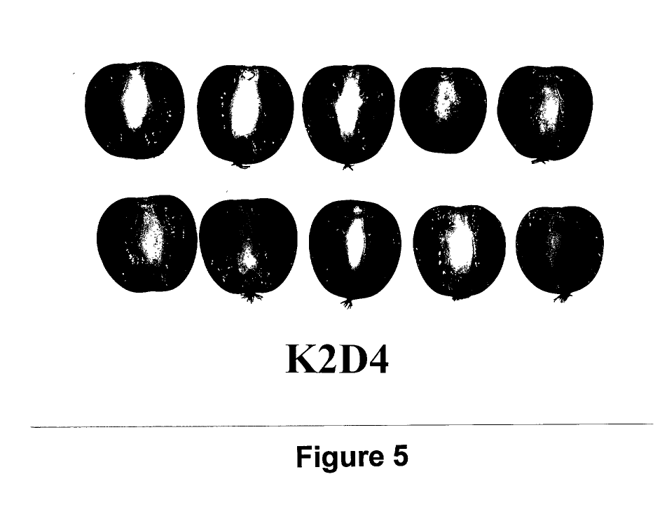

FIG. 4 shows fruit of the new variety in cross-section.

FIG. 5 shows fruit of the new variety in longitudinal section.

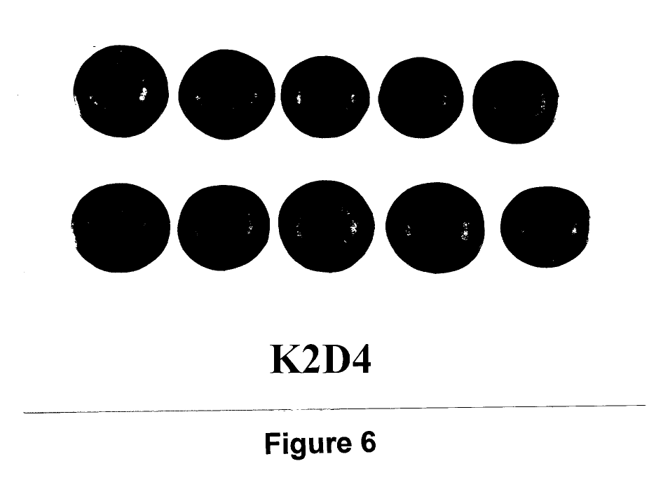

FIG. 6 shows the stem end and stem end cavity of the fruit from the new variety.

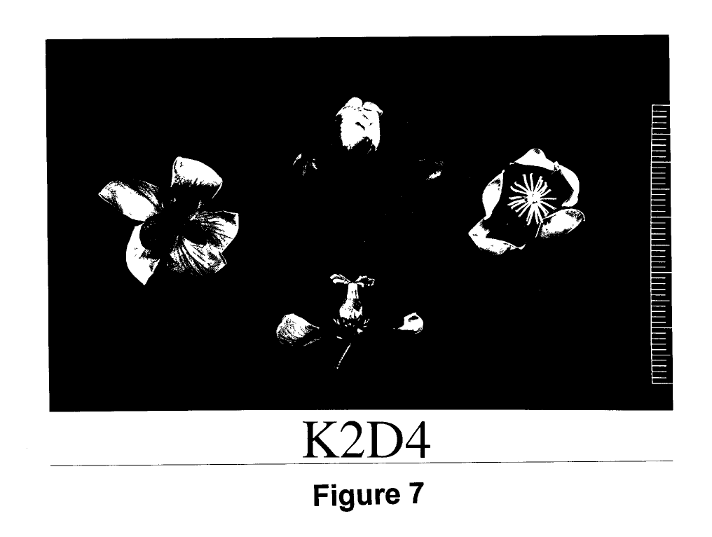

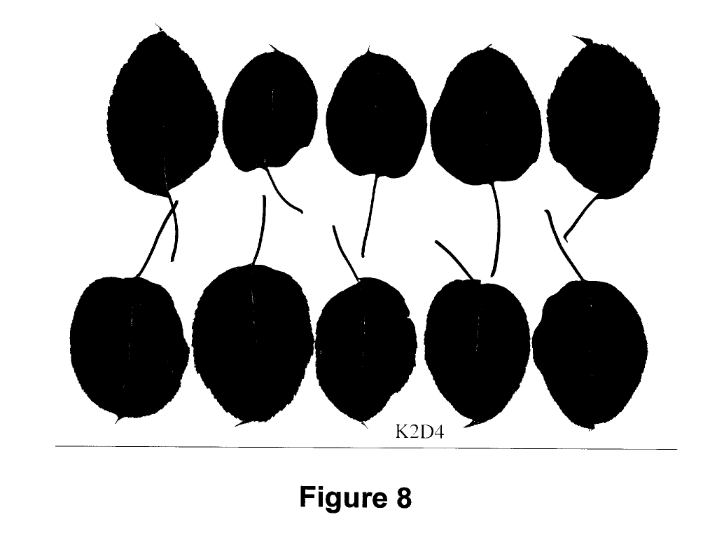

FIG. 7 shows flowers of the new variety.

Photographs of fruit from the new variety were taken after the normal harvest date. Fruit skin color may vary depending upon extent of exposure to direct sunlight.

### COMPARISON TO CLOSEST VARIETY

The distinctive characteristics of this new Kiwi variety, described in detail below, were observed in 2000 at Te Puke, New Zealand. The age of the plants was 5 years from planting cutting-grown plants. ‘HORTGEM TAHI’ is a new type of kiwi with no close varieties available in New Zealand. The A. deliciosa variety ‘Hayward’ and the A. chinensis variety ‘Hort16A’ are different in most characters and so do not provide a useful comparison. A. arguta varieties are grown in other countries, e.g. ‘Annanaskaya’ (unpatented) in U.S.A., but are unavailable in New Zealand.

The fruit of ‘HORTGEM TAHI’ is spheriod in shape with a rounded distal end. ‘HORTGEM TAHI’ fruit has a relatively large core proportionate to the surrounding pericarp. The flesh is green. The skin is without hairs and is a shiny green. ‘HORTGEM TAHI’ fruit have a high dry matter content at harvest and are sweet tasting when ripe. Fruit can be cool stored for 12 weeks and retain eating quality.

### DESCRIPTION OF THE NEW VARIETY

The new variety ‘HORTGEM TAHI’ is pistallate, with morphologically perfect but functionally imperfect flowers, i.e. the flowers produce only sterile pollen and thus require a pollenizer for fruit production. Three specific unpatented A. arguta pollenizers, designated B4G4, E4H4, K1J6, have been developed for ‘HORTGEM TAHI’. Characteristics of the new variety include a short period (˜3 months) between flowering and harvest, early harvest, small spheroid fruit with green hairless edible skin, green flesh and a sweet aromatic flavor. The following description are features described as they appear at the Public Research Center, New Zealand

### TABLE OF CHARACTERISTICS

Horticultural terminology is used in accordance with revised UPOV guidelines for kiwi.

All dimensions in millimeters, weights in grams (unless otherwise stated).

Color chart: RHS Color Chart, the Royal Horticultural Society, London (3^{rd} ed. 1995).

PLANT
(Measurements from samples of 20, unless otherwise stated)

| MATURE PLANT | |
|---|---|
| Sex expression | female (flowers imperfect) |
| Ploidy | tetraploid (2n = 2x = 116) |
| Vigour | strong |

YOUNG SHOOT:

| | |
|---|---|
| Hairs | present |
| Density of hairs | medium |
| Type of hairs | tomentose |
| Anthocyanin coloration | absent or very weak |
| of growing tip | |
| Anthocyanin coloration | absent or very weak |
| of leaf axil | |

STEM

| | |
|---|---|
| Coloration of leaf axil | absent or very weak |
| Diameter | thin; between about 7 to 8 |
| | mm.; Range: between about 4.2 |
| | to 12.2 |
| Dormant bud diameter | small; about 2.2 mm. (between |
| | about 0.7 and 4.0) |
| Color on upper side of shoot | near red brown |
| Character of bark | smooth |
| hairs | Absent |
| Consicuousness of lenticels | Conspicuous |
| Number of lenticels | Many |
| Color of lenticels | Near light red brown |
| Size of bud support | Medium |
| Visibility of bud (dormant | visible |
| canes) | |
| Number of hairs visible on | absent |
| bud (dormant canes) | |
| Leaf scar | deep |
| Stem color on exposed side | Between 166B and 176A |

LEAF (MATURE)

| | |
|---|---|
| General shape of blade | broad; ovate |
| Length | about 102.3 mm. (Between 82 |
| | and 126) |
| Width | about 75.1 mm. (Between 62 |
| | and 89) |
| Petiole length | about 57.7 mm. (Between 38 |
| | and 93) |
| Shape of tip of blade | Caudate |
| Shape of base of blade | rounded |
| Arrangement of leaf bases | far apart |
| Puckering/blistering on | absent or very weak |
| upper side of blade | |
| Margin | ciliate |
| Color of upper side of blade | between 137A and 139A |
| (in mature leaf after petal | |
| fall) | |
| Glossiness of upper surface | medium |
| of blade | |
| Color of lower side of blade | light green between 138B and |
| (in mature leaf after petal | 146B |
| fall) | |
| Glaucosity (lower side of | absent |
| blade) | |
| Hans on petiole | absent or very weak |
| Density of hairs on petiole | absent or very sparse |
| Anthocyanin coloration on | medium |
| upper side of petiole | |

FLOWER

| | |
|---|---|
| Inflorescence | Predominant number of flowers - |
| | usually one |
| PEDICEL | |
| length | short; about 29.8 mm (between |
| | 24.9 and 33.8) |
| Hairs | sparse |
| length of hairs | very short |
| number of sepals | About 5.1 (between 5 and 6) |
| diameter (terminal or | small, about 28.2 mm. |
| king flower when fully | (Between 23.2 and 31.2) |
| open) | |
| Petal length | about 14.5 mm. (Between 12.7 |
| | and 15.7) |
| Petal width | about 13.0 mm. (Between 11.0 |
| | and 14.6) |
| petal length/width ratio | about 1.12 (Between 1.02 and |
| | 1.33) |
| Mean number of petals | About 5.4 (Between 5 and 7) |
| per flower | |
| Number of flowers with | About 1 out of 20 (13 with 5, |
| more than six petals | 6 with 6) |
| Arrangement of petals | apart |
| Petal shoulder | absent |
| Color of main body of | near 157A |
| petals (when fully open) | |
| Type of coloration of | uniform color over whole |
| petals | petal |
| REPRODUCTIVE ORGANS | |
| Number of stamens | about 44.4 (Between 40 and |
| | 50) |
| Length of stamen, | about 4.2 mm (Between 3.6- |
| filaments | 5.0) |
| Length of anthers | about 3.7 mm (Between 3.2- |
| | 4.2) |
| Filament color | near light green |
| Anther color | Near grey black |
| Style number | about 20.7 (Between 18 and |
| | 24) |
| Length of styles | about 4.4 mm. (Between 4.1 |
| | and 5.2) |
| Attitude of styles | horizontal |
| Curvature of styles | weak |
| Length of ovary | about 6.8 mm. (Between 6.0 |
| | and 7.4) |
| Amount of hair on ovary | absent |
| Color of ovary | near 145A |

FRUIT

| | |
|---|---|
| Overall size | small, about 11.1 g. |
| | (Between 9.5 and 12.8) |
| Length | about 26.5 mm. (Between 23.6 |
| | and 29.4) |
| Width | |
| Maximum | about 26.8 mm. (Between 24.3 |
| | and 30.3) |
| Minimum | about 24.3 mm. (Between 21.8 |
| | and 28.0) |
| CORE DIAMETER | |
| Maximum | about 9.3 mm. (Between 6.9 |
| | and 11.4) |
| Minimum | about 5.0 mm. (Between 3.3 |
| | and 6.6) |
| Locule number | about 20.7 (Between 18 and |
| | 26) |
| Peduncle length | about 29.5 mm. (Between 23.6 |
| | and 34.0) |
| Peduncle width | about 2.0 mm. (Between 1.5 |
| | and 2.5) |
| General shape | spheroid |
| Cross section at median | oblate |
| General shape of stylar end | rounded |
| Shape of shoulder on stalk | square |
| end | |
| Skin color at harvest (fruit | near medium green |
| still hard) | |
| Skin color change during | absent |
| ripening | |
| Skin color at maturity for | between 141c and 143c |
| consumption | |
| Fruit hairs | absent |
| FRUIT CORE | |
| Diameter (at largest | large |
| diameter) | |
| Shape (in cross-section) | oblate |
| woody spike | absent |
| Outer pericarp color at | between 139B and 141B |
| maturity for consumption | |
| Inner pericarp col. | between 139B and 141B |
| (locules) at mature for | |
| consumption | |
| Color at maturity | near green white |
| Color at harvest | between near 150D and 157B |
| Sweetness (Brix level) at | about 20.2% (Between 17.2 and |
| maturity for consumption | 22.0) |
| Vitamin C content (45 fruit | about 51 mg./100 g. fresh |
| sample) | weight (Between 37 and 67) |
| Seed color at maturity (in | between 172B and 175C |
| flesh) | |
| seed color when dry | Between 172B and 175C |

EVENTS

| | |
|---|---|
| Time of vegetative budbreak | about late August |
| Time of beginning of | about mid-November |
| flowering | |
| Time of maturity for harvest | between about late February |
| (at nominated Brix level) | and early March |

### HORTICULTURAL CHARACTERISTICS

Details below relate to observations made on plants growing at Te Puke Research Centre, New Zealand. These plants were cutting-grown.

### CROPPING

Young vines of the new variety are precocious, beginning to bear in their second year and are expected to reach full capacity at about 7 years. The storage life of the fruit of the new variety is about 10 to 12 weeks at 0° C., if stored in unventilated containers.

### FRUIT SIZE

Data from harvesting all fruit from 6 vines in late February 1999 is as follows:

Mean fruit weight: 10.8 g

Maximum: 12.6 g

Minimum: 9.7 g

Mean fruit number: 2087

Maximum: 3610

Minimum: 802

Mean yield: 21.96 kg

Maximum: 35.14 kg

Minimum: 8.42 kg

These vines were about 3 years old. It is expected that for mature, well-managed vines mean fruit weight will be about 10 g., mean fruit number about 5000 fruit, and mean yield per vine about 50 kg.

We claim:

1. A new and distinct kiwi plant of the species A. arguta substantially as described and illustrated, characterized by small spheroid fruit with green hairless edible skin and green flesh with a sweet aromatic flavor.
